(19) United States
(12) Patent Application Publication
(10) Pub. No.: US 2026/0035713 A1
(43) Pub. Date: Feb. 5, 2026

(54) APPLICATION OF PLANT TIM PROTEIN, TIM GENE ENCODING THE SAME OR HOMOLOGOUS GENE OF THE SAME IN PLANT RESISTANCE TO VIRUSES, METHOD FOR OBTAINING VIRUS-RESISTANT PLANT AND METHOD FOR IDENTIFYING ANTIVIRAL CAPABILITY

(71) Applicant: Jiangsu Academy of Agricultural Sciences, Jiangsu (CN)

(72) Inventors: Xiaoyan Cui, Jiangsu (CN); Hao He, Jiangsu (CN); Fangfang Li, Jiangsu (CN); Wei Zhang, Jiangsu (CN); Xin Chen, Jiangsu (CN)

(73) Assignee: Jiangsu Academy of Agricultural Sciences, Jiangsu (CN)

(21) Appl. No.: 18/916,759

(22) Filed: Oct. 16, 2024

(30) Foreign Application Priority Data
Aug. 1, 2024 (CN) 202411049551.7

(51) Int. Cl. C12N 15/82; C12Q 1/6895; G01N 33/68

(52) U.S. Cl. CPC C12N 15/8283; C12N 15/8218; C12Q 1/6895; G01N 33/68; C12Q 2600/13; G01N 2333/415

(57) ABSTRACT

Disclosed are an application of a plant TIM protein, a TIM gene encoding the plant TIM protein or a homologous gene of the same in plant resistance to viruses. Further disclosed is a method for obtaining virus-resistant plant, where the method involves downregulating the expression or activity of the plant TIM protein. Further disclosed is a method for identifying whether the plants obtained by the above method has antiviral capability, to determine whether the plant contains TIM protein or TIM genes through identification. The present disclosure introduces a gene editing vector and a gene silencing vector carrying a target gene into target plant using a transgenic method, so as to obtain homozygous plant with GmTIM gene knockout in soybean and plant with down-regulated expression of NbTIM in Nicotiana benthamiana, which are capable of significantly inhibiting viral infection.

### CROSS-REFERENCE TO RELATED APPLICATION

This application claims the priority benefit of China application serial no. 202411049551.7, filed on Aug. 1, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Sep. 30, 2024, is named 149494-US-sequence listing and is 28,672 bytes in size.

### BACKGROUND

### Technical Field

The present disclosure belongs to the application field of agricultural science and technology, and particularly relates to an application of plant TIM protein, or TIM gene encoding the same or homologous gene of the same in plant resistance to viruses.

### Description of Related Art

Soybean mosaic virus (SMV) is one of the most common viral diseases in soybean production areas around the world. Under natural conditions, SMV can cause economic losses ranging from 35% to 50%, and may even reach as high as 50% to 100% in severe cases, apart from directly causing significant reduction in soybean yield and germplasm decline, SMV infection also greatly reduces the immunity of soybean to other pathogens (Arif and Hassan, 2002). Therefore, SMV has become a major factor restricting the soybean production. At present, no safe and effective chemical agents are available for the control of SMV diseases, therefore, developing disease-resistant varieties is a most economically effective way to control the SMV diseases.

SMV is an RNA virus belonging to the genus Potyvirus, featuring quick strain variations and being easy to bypass host resistance genes. Therefore, when a single resistant variety is planted over a very large area, the resistance dominated by dominant resistance genes can be easily broken by the evolution of pathogens, leading to large-scale outbreak of the disease, therefore, breeders have to seek new resistance strategies due to such vicious cycle. Recessive resistance genes are essential genes for survival (replication, movement, assembly, and the like) of plant viruses. Approximately 200 antiviral genes are known, about half of these genes are recessively inherited, indicating that recessive resistance strategies are more common in the process of plant-virus interaction compared with other pathogens, and recessive resistance tends to be more durable (Hashimoto et al., 2016; Truniger and Aranda, 2009). Obtaining genetic resistance by genetically modifying and mutating host genes that are essential for virus infection (recessive resistance genes) has become a new strategy in antiviral breeding. For example, translation initiation factor eIF4E and homologous gene of the same that have been studied more thoroughly are essential host factors for many important crop-infecting Potyviruses, and have been used as important recessive resistance genes in gene-editing breeding (Robaglia and Caranta, 2006; Bastet et al., 2017).

A mitochondrial translocase subunit TIM family protein forms major protein transport complexes on a mitochondrial inner membrane, such as mitochondrial inner membrane carrier protein translocase (TIM22 complex) and mitochondrial inner membrane presequence translocase (TIM23 complex). The TIM23 complex regulates the insertion of protein with presequences into the inner membrane, a Tim23 subunit thereof is responsible for transporting the protein into a matrix and is also capable of inserting some protein into the inner membrane (Bauer et al., 1996; Donzeau et al., 2000); and the TIM22 complex is responsible for the transport and insertion of hydrophobic membrane protein, and a Tim22 subunit thereof is responsible for inserting mitochondrial metabolite transport protein, such as ADP/ATP and phosphate transport protein, into the inner membrane (Bauer et al., 2000; Duncan et al., 2013).

At present, no reports on the application of TIM protein and TIM genes in plant resistance to viruses are available.

### SUMMARY

A first technical problem to be solved by the present disclosure is to provide an application of a TIM gene or homologous gene of the same in plant resistance to viruses.

A second technical problem to be solved by the present disclosure is to provide a method for obtaining virus-resistant plant.

A third technical problem to be solved by the present disclosure is to provide a method for identifying whether the plant obtained by the foregoing method has antiviral capability.

The present disclosure can make the plants resistant to viruses by silencing TIM genes of the Nicotiana benthamiana or knocking out TIM genes of soybeans, this invention enables plants to develop resistance, exhibiting broad-spectrum disease resistance.

Technical solutions: in order to achieve the above objectives, the present disclosure provides an application of plant TIM protein, or TIM gene encoding the same or homologous gene of the same in plant resistance to viruses.

Specifically, an amino acid sequence of the plant TIM protein is shown in SEQ ID NO. 1 or SEQ ID NO. 26, and a nucleotide sequence of the TIM gene is shown in SEQ ID NO. 2 or SEQ ID NO. 5.

The present disclosure provides a method for obtaining virus-resistant plant, the method includes: downregulating the expression or activity of the plant TIM protein, where the amino acid sequence of the plant TIM protein is shown in SEQ ID NO. 1 or SEQ ID NO. 26; and preferably, a method for downregulating the expression or activity of the plant TIM protein involves knocking out or silencing the TIM gene in plant genomes, and the nucleotide sequence of the TIM gene is shown in SEQ ID NO. 2 or SEQ ID NO. 5.

Specifically, a method for downregulating involves knocking out the GmTIM gene to interfere with the expression of the GmTIM gene, and preferably, a nucleotide sequence of the GmTIM gene after knockout is shown in SEQ ID NO. 3 or SEQ ID NO. 4.

Specifically, the virus includes soybean mosaic virus (SMV), turnip mosaic virus (TuMV), tobacco mosaic virus (TMV) of Tobamovirus, and potato virus X (PVX) of Potexvirus.

The plants include but are not limited to soybean or Nicotiana benthamiana.

Specifically, the method for downregulating further includes silencing the homologous gene NbTIM by constructing a dsRNA transcript of the gene, a nucleotide sequence of the NbTIM is shown in SEQ ID NO. 5, and a sequence of the transcript dsRNA thereof is shown in SEQ ID NO. 6.

The present disclosure further includes a method for identifying whether the plant obtained by the foregoing method has antiviral capability, to determine whether the plant contains TIM protein or TIM genes through identification.

Specifically, the method for identifying is detected by RT-qPCR or Western blotting.

Specifically, sequences of target detection primers for the turnip mosaic virus (TuMV) are shown in SEQ ID NO. 7 and SEQ ID NO. 8, sequences of target detection primers for the tobacco mosaic virus (TMV) of Tobamovirus are shown in SEQ ID NO. 9 and SEQ ID NO. 10, sequences of target detection primers for the potato virus X (PVX) of Potexvirus are shown in SEQ ID NO. 11 and SEQ ID NO. 12, and sequences of target detection primers for the soybean mosaic virus (SMV) are shown in SEQ ID NO. 13 and SEQ ID NO. 14.

Beneficial effects: compared with the prior art, the present disclosure has the outstanding effects: the present disclosure discovers for the first time the functions of plants, including soybean TIM genes and tobacco TIM genes, knockout of the soybean TIM genes and silencing of the tobacco TIM genes can make the plants resistant to viruses, and have broad-spectrum disease resistance.

The application of TIM gene silencing/knocking out the plants TIM genes in regulating plant viral resistance and the method for cultivating transgenic plants will be further described below in conjunction with the description of drawings and specific embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A to FIG. 1E show that knocking out GmTIM makes soybean resistant to soybean mosaic virus (SMV); specifically, (FIG. 1A) sequencing demonstrates that GmTIM is knocked out from knockout plant; (FIG. 1B) phenotypes of WT and GmTIM knockout plants 10 days after being inoculated with SMV, the WT plant shows mosaic symptoms, and the GmTIM knockout plant does not have any symptoms; (FIG. 1C) phenotypes of the WT and GmTIM knockout plants 30 days after being inoculated with SMV, and the GmTIM knockout plant does not develop disease yet; (FIG. 1D) RT-qPCR detection of a RNA level of SMV in the plants; and (FIG. 1E) detection of accumulation of SMV protein in the plants by Western blotting.

FIG. 2A to FIG. 2D show that silencing NbTIM makes Nicotiana benthamiana resistant to turnip mosaic virus (TuMV); specifically, (FIG. 2A) RT-qPCR detection of an expression level of NbTIM in NbTIM-RNAi transgenic plants; (FIG. 2B) phenotypes of the transgenic plants 14 days after being inoculated with TuMV-GFP; (FIG. 2C) RT-qPCR detection of a RNA level of TuMV in the plants; and (FIG. 2D) detection of accumulation of SMV proteins in the plants by Western blotting.

FIG. 3A to FIG. 3D shows that silencing NbTIM makes Nicotiana benthamiana resistant to tobacco mosaic virus (TMV); specifically, (FIG. 3A) RT-qPCR detection demonstrates that NbTIM in NbTIM-RNAi transgenic plants is silenced; (FIG. 3B) phenotypes of WT and NbTIM-RNAi knockout plants 10 days after being inoculated with TMV-GFP; (FIG. 3C) RT-qPCR demonstrates that a RNA level of TMV in the NbTIM-silenced plant is significantly lower than that in the WT plant; and (FIG. 3D) Western blotting demonstrates that accumulation of TMV protein in the NbTIM-silenced plants is significantly lower than that in the WT plant.

FIG. 4 shows that silencing NbTIM makes Nicotiana benthamiana resistant to potato virus X (PVX); specifically, (A) RT-qPCR detects expression levels of WT and NbTIM in NbTIM-RNAi transgenic plant; (B) phenotypes of WT and NbTIM-RNAi knockout plants 14 days after being inoculated with PVX-GFP; (C) RT-qPCR demonstrates that a RNA level of PVX in the NbTIM-silenced plant is significantly lower than that in the WT plant; and (D) Western blotting demonstrates that accumulation of PVX protein in the NbTIM-silenced plant is significantly lower than that in the WT plant.

### DESCRIPTION OF THE EMBODIMENTS

Embodiments of the present disclosure will be described in detail below with reference to the examples, and the following examples are only used to illustrate the present disclosure. Any example without indicating specific conditions shall be performed according to conventional conditions or conditions recommended by corresponding manufacturer. Reagents or instruments used without indicating manufacturers are conventional products commercially available.

### Example 1: Knockout of GmTIM can Make Plant Resistant to Viruses

Target primers were designed as follows based on a sequence of soybean GmTIM (as shown in SEQ ID NO. 2): 5′-CGCAGCAGAACGTAGATACTGTTTTAGAGCTAGAAATAGCAAG-3′ (SEQ ID No. 15) and 5′-AGTATCTACGTTCTGCTGCGAATCCATATGTTTTCCTGGGAC-3′ (SEQ ID No. 16).

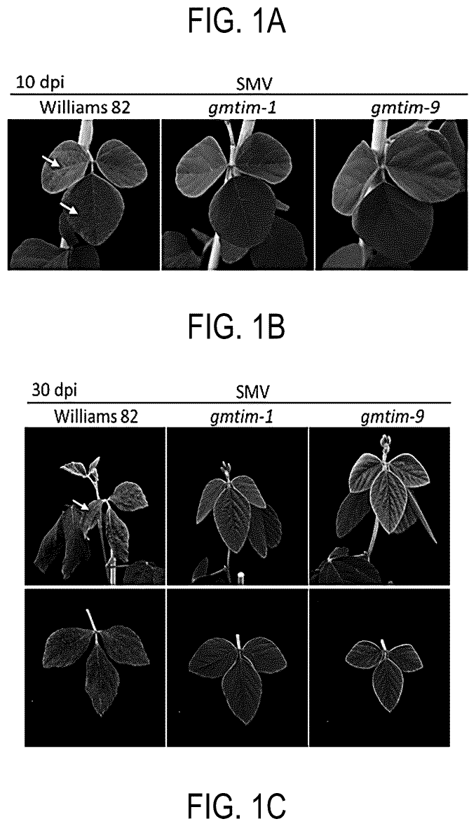

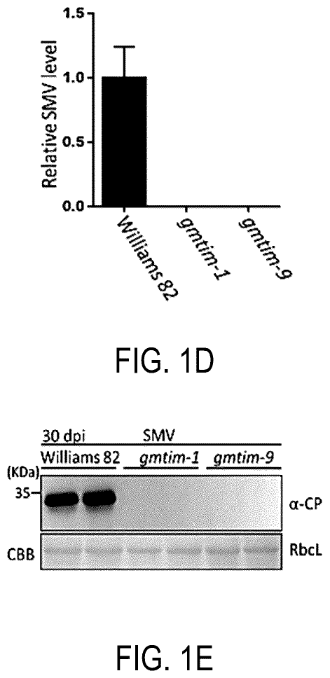

Target fragments were recombined into pGmUbi-Cas9, a CRISPR-Cas9 vector according to specific methods and same conditions as stated in the reference (Zhang P, Du H, Wang J, Pu Y, Yang C, Yan R, Yang H, Cheng H, Yu D. Multiplex CRISPR Cas9-mediated Metabolic Engineering Increases Soya Bean Isoflavone Content and Resistance to Soya Bean Mosaic Virus, Plant Biotechnol J. 2020 June; 18 (6): 1384-1395. doi: 10.1111/pbi.13302.), to obtain a pGmUbi-Cas9-GmTIM vector. 1 μg of the pGmUbi-Cas9-GmTIM vector and 100 μl of Agrobacterium competent cells (strain EHA105) were mixed and then transferred into a clean electroporation cuvette, transformation was carried out using an electroporation device at 2500 V, a pre-cooled LB liquid medium was immediately added, and recovered at 28° C. for 2 h before being applied in a resistance medium (a LB medium with Kanamycin resistance) to grow for 48 h, and screening was performed to obtain positive Agrobacterium. A positive single colony were picked, and transferred into a liquid resistance medium (a LB medium with rifampicin and kanamycin resistance) and cultured overnight at 28° C. and 200 rpm; OD600 of the Agrobacterium was adjusted to be 1.0, and then inoculated into soybean plants to obtain infected soybean cotyledon nodes, which were differentiated and cultured to obtain callus, rooting culture was then carried out to obtain seedlings, and the seedlings were transferred for continuous culture to obtain regenerated soybean plants of T0 generation. Part of leaf samples were collected, DNA of the regenerated soybean plants was extracted using a CTAB method, the DNA was then used as a template, a 700 bp fragment was amplified with a primer pair 5′-TATCACCGATAAGCCCACTGAGGC-3′ (SEQ ID No. 17) and 5′-CACCATCATCAACAACAAATCAGTAGCT-3′ (SEQ ID No. 18), and sequencing was performed to determine whether the regenerated soybean plants had been edited. Sequencing results showed that 4-base deletion (as shown in SEQ ID NO. 3) and 14-base deletion (as shown in SEQ ID NO. 4) near a target sequence of two gene-edited strains (GmTIM-1 and GmTIM-9) were respectively identified, as shown in FIG. 1A. Wild-type plants (Williams 82) and GmTIM-edited plants were inoculated with SMV by friction. 10 days later, the wild-type plants showed typical mosaic symptoms, but the gene-edited strains showed no symptoms (FIG. 1B); and 30 days later, the wild-type plants exhibited typical mosaic and rugose leaves, but the gene-edited strains did not have any symptoms yet (FIG. 1C). Both RT-qPCR and Western blotting results indicated that viral RNA and coat proteins in the soybean with GmTIM knockout were negative (FIG. 1D to FIG. 1E), indicating that knockout of GmTIM made soybean plants resistant to viruses. The foregoing results indicated that knockout of GmTIM made plants resistant to viruses.

### Example 2: Silencing NbTIM can Make Plant Resistant to Viruses

An RNA interference vector was constructed with NbTIM genes as target genes (SEQ ID No. 5) using RNA interference (RNAi) technology.

Nicotiana benthamiana RNA was extracted, reverse transcription was performed to obtain cDNA, the cDNA was used as a template, and nucleotide sequences from positions 4 to 437 of the NbTIM transcript (SEQ ID No. 6) was amplified by PCR with two pairs of primers as a complementary DNA double strand of a hairpin structure. A first pair of primers was 5′-CGGGATCCGATGATGCTGCAGAGCTAAG-3′ (SEQ ID No. 19) and 5′-GGGCCCCTCCTTGCCATTGTCAGTTC-3′ (SEQ ID No. 20), and a reaction system was 25 μL, including 12.5 μL of PCR Mix, 10 ng of cDNA template, 1 μL of each of the primers, with a balance being water to make up to 25 μL. PCR conditions were as follows: pre-denaturation at 94° C. for 3 min; 30 cycles of 94° C. for 30 s, 55° C. for 30 s, and 72° C. for 1 min; followed by a final extension at 72° C. for 7 min; and a resulting PCR product was fNbTIM (forward). In order to facilitate vector construction, restriction enzyme sites for BamHI and ApaI were added at 5′ and 3′ ends of fNbTIM through PCR. A second pair of primers was 5′-ACGCGTCGACCTACTACGACGTCTCGATTCC-3′ (SEQ ID No. 21) and 5′-CGACGCGTGAGGAACGGTAACAGTCAAGA-3′ (SEQ ID No. 22). A resulting PCR product was rNbTIM (reversed). The fNbTIM was double-digested with BamHI and ApaI (reacted at 37° C. for 30 min), and the rNbTIM was double-digested with SalI and MluI, and connected into a pCambia2301 vector subjected to double-digested with BamHI and ApaI (22° C. for 15 min), to obtain an RNA interference vector pCambia2301-NbTIM.

1 μg of the RNA interference vector pCambia2301-NbTIM and 100 μl of Agrobacterium competent cells (strain EHA105) were mixed and then transferred into a clean electroporation cuvette, transformation was carried out using an electroporation device at 2500 V, a pre-cooled LB liquid medium was immediately added, and recovered at 28° C. for 2 h before being applied in a resistance medium to grow for 48 h, and screening was performed to obtain positive Agrobacterium. A positive single colony were picked, and transferred into a liquid resistance medium, and cultured overnight at 28° C. and 200 rpm; OD600 of the Agrobacterium was adjusted to be 1.0, and then inoculated into Nicotiana benthamiana to obtain infected Nicotiana benthamiana cotyledon nodes, which were differentiated and cultured to obtain callus, rooting culture was then carried out to obtain seedlings, and the seedlings were transferred for continuous culture to obtain regenerated Nicotiana benthamiana plants of T0 generation, and primer pairs 5′-CGGGATCCGATGATGCTGCAGAGCTAAG-3′ (SEQ ID No. 19) and 5′-ACGCGTCTGTAATCAATCCAAATGTAAGATCAATG-3′ (SEQ ID No. 23) were used to detect whether marker genes had been integrated into the genome; in addition, primer pairs 5′-CAGTTGGTGGATTCGTTGC-3′ (SEQ ID No. 24) and 5′-TTGTCAGTTCTCGTTGTCGC-3′ (SEQ ID No. 25) were used to perform fluorescence quantitative PCR (RT-qPCR) detection of target genes in the regenerated Nicotiana benthamiana plants transformed with the RNAi vector to determine whether a transcription level of the target gene was downregulated due to silencing.

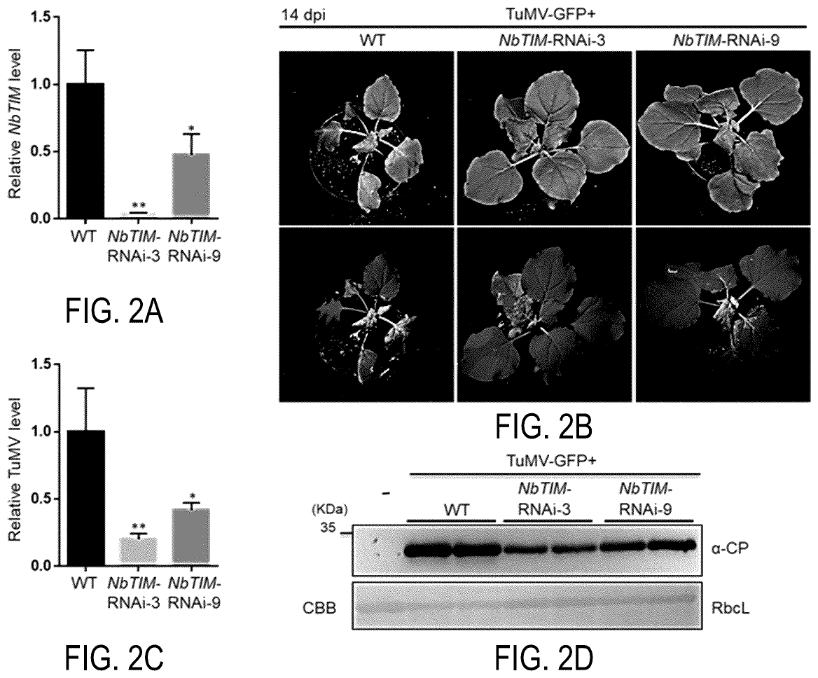

In order to identify the role of TIM in viral infection, two NbTIM-silenced lines (Line 3 and Line 9) with effective NbTIM silencing were selected for further experiments. Results of RT-qPCR detection of an expression level of NbTIM in the NbTIM-RNAi transgenic plants were shown in FIG. 2A; compared with the wild-type plants, NbTIM-silenced plants delayed disease onset and milder symptoms, with less green fluorescence in systemic leaves, the NbTIM-silenced plants only showed limited virus spread to systemic leaves and were in good condition when the wild-type plants had begun to die. Samples from a same part of the systemic leaves were collected for further extraction of RNA and total proteins. RT-qPCR results showed that a level of viral RNA in the NbTIM-silenced plants was significantly lower than that in the wild-type plants (FIG. 2C), Western blotting was adopted to detect the accumulation of TuMV proteins, and 14 days after TuMV-GFP inoculation into both the wild-type and NbTIM-RNAi transgenic plants, results of western blotting further demonstrated that the accumulation of coat proteins in the NbTIM-silenced plants was significantly lower than that in the control plants (FIG. 2D).

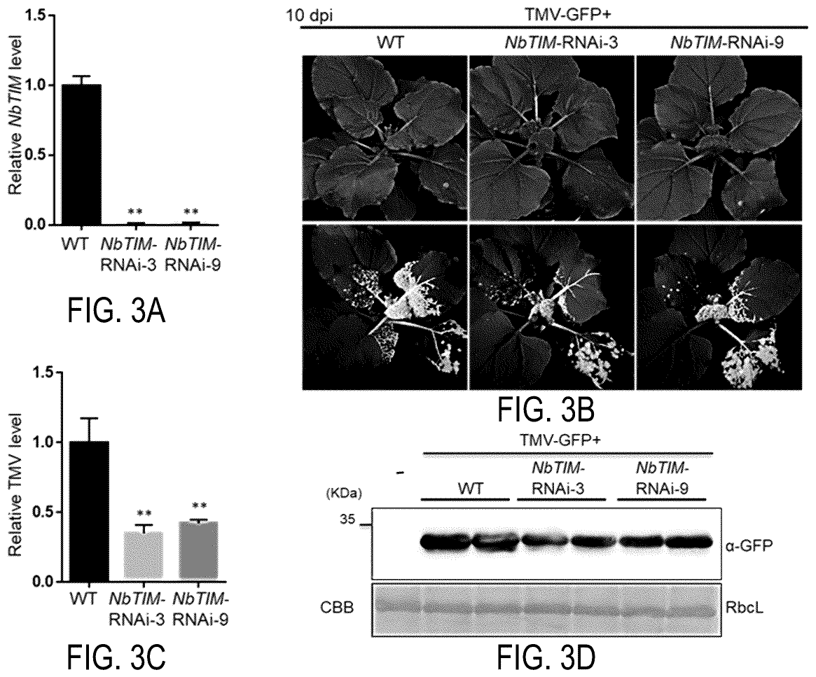

10 days after TMV-GFP (provided by the Functional Genomics of Crop Pathogens, Institute of Plant Protection, Chinese Academy of Agricultural Sciences) was used to infect both the wild-type (wild-type Nicotiana benthamiana leaves) and NbTIM-RNAi plants, the NbTIM-silenced plants showed milder symptoms, and results were shown in FIG. 3A to FIG. 3D.

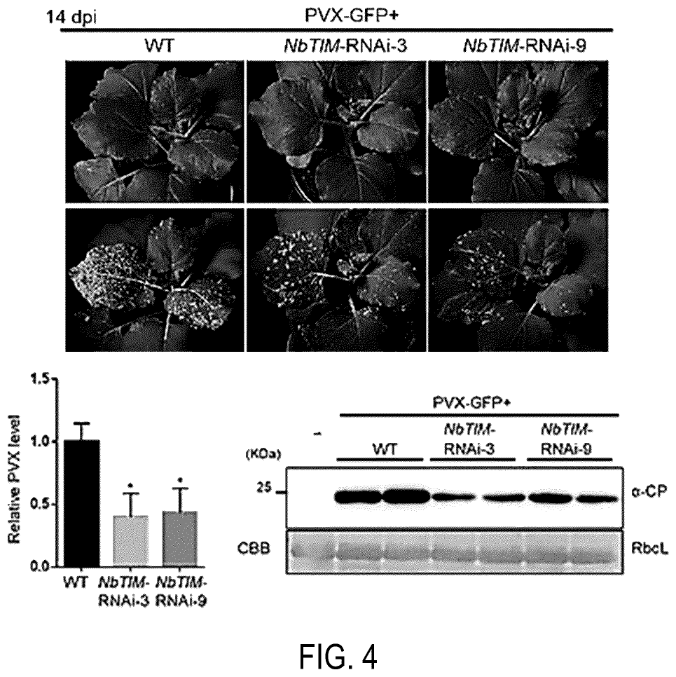

14 days after PVX-GFP (provided by the Plant and Virus Interaction Group, Institute of Plant Protection, Chinese Academy of Agricultural Sciences) was used to infect both the wild-type (wild-type Nicotiana benthamiana leaves) and NbTIM-RNAi plants, the NbTIM-silenced plants showed milder symptoms, and results were shown in FIG. 4.

The foregoing results indicated that silencing of TIM in tobacco conferred resistance to viruses from different genera, exhibiting a broader spectrum.

What is claimed is:

1. An application of a plant TIM protein, a TIM gene encoding the plant TIM protein or a homologous gene of the same in plant resistance to viruses.

2. The application according to claim 1, wherein an amino acid sequence of the plant TIM protein is shown in SEQ ID NO. 1 or SEQ ID NO. 26, and a nucleotide sequence of the TIM gene is shown in SEQ ID NO. 2 or SEQ ID NO. 5.

3. A method for obtaining a virus-resistant plant, comprising:
downregulating an expression or activity of a plant TIM protein, wherein an amino acid sequence of the plant TIM protein is shown in SEQ ID NO. 1 or SEQ ID NO. 26; and
a downregulating method of the expression or activity of the plant TIM protein includes knocking out or silencing a TIM gene in plant genomes, and a nucleotide sequence of the TIM gene is shown in SEQ ID NO. 2 or SEQ ID NO. 5.

4. The method according to claim 3, wherein the downregulating method includes knocking out the TIM gene to interfere with the expression of the TIM gene, and a nucleotide sequence of the TIM gene after knockout is shown in SEQ ID NO. 3 or SEQ ID NO. 4.

5. The method according to claim 3, wherein the virus comprises soybean mosaic virus (SMV), turnip mosaic virus (TuMV), tobacco mosaic virus (TMV) of Tobamovirus, and potato virus X (PVX) of Potexvirus.

6. The method according to claim 3, wherein the virus-resistant plant comprises soybean or Nicotiana benthamiana.

7. The method according to claim 3, wherein the downregulating method further comprises silencing a homologous gene NbTIM by constructing a dsRNA transcript of the homologous gene NbTIM, a nucleotide sequence of the homologous gene NbTIM is shown in SEQ ID NO. 5, and a sequence of the dsRNA transcript of the homologous gene NbTIM is shown in SEQ ID NO. 6.

8. A method for identifying whether a virus-resistant plant obtained by the method according to claim 3 has an antiviral capability, comprising determining whether the virus-resistant plant contains a TIM protein or a TIM gene through an identification.

9. The method according to claim 8, wherein the method for identifying comprises RT-qPCR or western blotting.

10. The method according to claim 9, wherein sequences of target detection primers for turnip mosaic virus (TuMV) are shown in SEQ ID NO. 7 and SEQ ID NO. 8, sequences of target detection primers for tobacco mosaic virus (TMV) of Tobamovirus are shown in SEQ ID NO. 9 and SEQ ID NO. 10, sequences of target detection primers for potato virus X (PVX) of Potexvirus are shown in SEQ ID NO. 11 and SEQ ID NO. 12, and sequences of target detection primers for soybean mosaic virus (SMV) are shown in SEQ ID NO. 13 and SEQ ID NO. 14.


SEQUENCE LISTING (26 sequences)

SEQ ID NO: 1 - protein, 160 aa, synthetic construct
MVDAAERRYLEDDDSSLMKTIKGATTGLVAGTIWGTVVATWYDVPRVERNVALPGLIRTIRMMGNYGLTFAAIGGVYIGVEQLVQNYRMKRDLVNGAVGGFVAGATILGYRGRSIKTALSAGSALAFTSAFLDFGDQTLKHDAGKEYAAYTTKKRPSTDA

SEQ ID NO: 2 - DNA, 483 bp, synthetic construct
  source 1..483: mol_type = other DNA
atggtggacgcagcagaacgtagatacttggaggatgatgattcatctctcatgaaaactatcaagggcgcaacaactggtttagtggctggaactatctggggtactgtagttgccacctggtatgatgtcccccgtgtcgagagaaatgttgctcttccagggctgattaggactatcaggatgatgggcaactatggtttgacctttgctgccataggaggagtctacataggtgtagagcagttggtgcagaactataggatgaagagggatttagtcaatggagctgttggcggatttgttgcaggtgcaactattctgggttacagaggaaggagcatcaaaactgcactttctgctggatcagcattggcattcacatctgcattccttgatttcggtgatcagacattaaaacatgatgctgggaaggagtatgcggcttacaccacaaagaaaagacccagtactgatgcataa

SEQ ID NO: 3 - DNA, 479 bp, synthetic construct
  source 1..479: mol_type = other DNA
atggtggacgcagcagaacgtacttggaggatgatgattcatctctcatgaaaactatcaagggcgcaacaactggtttagtggctggaactatctggggtactgtagttgccacctggtatgatgtcccccgtgtcgagagaaatgttgctcttccagggctgattaggactatcaggatgatgggcaactatggtttgacctttgctgccataggaggagtctacataggtgtagagcagttggtgcagaactataggatgaagagggatttagtcaatggagctgttggcggatttgttgcaggtgcaactattctgggttacagaggaaggagcatcaaaactgcactttctgctggatcagcattggcattcacatctgcattccttgatttcggtgatcagacattaaaacatgatgctgggaaggagtatgcggcttacaccacaaagaaaagacccagtactgatgcataa

SEQ ID NO: 4 - DNA, 470 bp, synthetic construct
  source 1..470: mol_type = other DNA
atggtggacgcagcttggaggatgatgattcatctctcatgaaaactatcaagggcgcaacaactggtttagtggctggaactatctggggtactgtagttgccacctggtatgatgtcccccgtgtcgagagaaatgttgctcttccagggctgattaggactatcaggatgatgggcaactatggtttgacctttgctgccataggaggagtctacataggtgtagagcagttggtgcagaactataggatgaagagggatttagtcaatggagctgttggcggatttgttgcaggtgcaactattctgggttacagaggaaggagcatcaaaactgcactttctgctggatcagcattggcattcacatctgcattccttgatttcggtgatcagacattaaaacatgatgctgggaaggagtatgcggcttacaccacaaagaaaagacccagtactgatgcataa

SEQ ID NO: 5 - DNA, 480 bp, synthetic construct
  source 1..480: mol_type = other DNA
atggatgatgctgcagagctaaggtattatgaagatgaggatacaccggggatgaaaacatttaaaggtgcaacaacaggtctagttgctggtactctttggggtgccattattgccacttggcatgatgtaccccgtgttgagagaagtgttgctcttcctggtttgattcggacattgaaaatgatgggaaaccatggggctacctttgctgcaattggtggagtctatataggggtagaacagttggtgcagaactataggatgaaaagggattttgttaatggagcagttggtggattcgttgctggtgcttctatactgggttacaaaggaaggagtatttcaacagctatctctgctggagcagcacttgcagtcacctctggggtaattgatggattaggtgcgacaacgagaactgacaatggcaaggagtactatccttacactacgaagaaaagagctgctgttgaatga

SEQ ID NO: 6 - DNA, 435 bp, synthetic construct
  source 1..435: mol_type = other DNA
gatgatgctgcagagctaaggtattatgaagatgaggatacaccagggatgaaaacatttaaaggtgcaaccacaggtctagttgctggtactctttggggtgccattattgccacttggcatgatgtaccccgtgttgagagaagtgttgctcttcctggtttgattcggacattgaaaatgatgggaaaccacggggctacctttgctgctattggtggagtctatataggggtagaacagctggtgcagaactataggatgaaaagggattttgttaatggagcagttggtggattcgttgctggtgcttctgtactgggttacaaaggaaggagtatttcaacagctatctctgctggagcagcacttgcagtcacctctggggtaattgatggattaggtgcgacaacgagaactgacaatggcaaggag

SEQ ID NO: 7 - DNA, 19 bp, synthetic construct
  source 1..19: mol_type = other DNA
cggaatgtgggtgatgatg

SEQ ID NO: 8 - DNA, 20 bp, synthetic construct
  source 1..20: mol_type = other DNA
ctgcctaaatgtgggtttgg

SEQ ID NO: 9 - DNA, 20 bp, synthetic construct
  source 1..20: mol_type = other DNA
cagttcaaggtcgttcccaa

SEQ ID NO: 10 - DNA, 20 bp, synthetic construct
  source 1..20: mol_type = other DNA
cccgctgacatcttcacatt

SEQ ID NO: 11 - DNA, 20 bp, synthetic construct
  source 1..20: mol_type = other DNA
gctaactggcaagcacaagg

SEQ ID NO: 12 - DNA, 19 bp, synthetic construct
  source 1..19: mol_type = other DNA
tgggcagcattcatttcag

SEQ ID NO: 13 - DNA, 20 bp, synthetic construct
  source 1..20: mol_type = other DNA
tgatgatgagcagatgggtg

SEQ ID NO: 14 - DNA, 21 bp, synthetic construct
  source 1..21: mol_type = other DNA
gggcttcagtgggtattcaat

SEQ ID NO: 15 - DNA, 43 bp, synthetic construct
  source 1..43: mol_type = other DNA
cgcagcagaacgtagatactgttttagagctagaaatagcaag

SEQ ID NO: 16 - DNA, 42 bp, synthetic construct
  source 1..42: mol_type = other DNA
agtatctacgttctgctgcgaatccatatgttttcctgggac

SEQ ID NO: 17 - DNA, 24 bp, synthetic construct
  source 1..24: mol_type = other DNA
tatcaccgataagcccactgaggc

SEQ ID NO: 18 - DNA, 28 bp, synthetic construct
  source 1..28: mol_type = other DNA
caccatcatcaacaacaaatcagtagct

SEQ ID NO: 19 - DNA, 28 bp, synthetic construct
  source 1..28: mol_type = other DNA
cgggatccgatgatgctgcagagctaag

SEQ ID NO: 20 - DNA, 26 bp, synthetic construct
  source 1..26: mol_type = other DNA
gggcccctccttgccattgtcagttc

SEQ ID NO: 21 - DNA, 31 bp, synthetic construct
  source 1..31: mol_type = other DNA
acgcgtcgacctactacgacgtctcgattcc

SEQ ID NO: 22 - DNA, 29 bp, synthetic construct
  source 1..29: mol_type = other DNA
cgacgcgtgaggaacggtaacagtcaaga

SEQ ID NO: 23 - DNA, 35 bp, synthetic construct
  source 1..35: mol_type = other DNA
acgcgtctgtaatcaatccaaatgtaagatcaatg

SEQ ID NO: 24 - DNA, 19 bp, synthetic construct
  source 1..19: mol_type = other DNA
cagttggtggattcgttgc

SEQ ID NO: 25 - DNA, 20 bp, synthetic construct
  source 1..20: mol_type = other DNA
ttgtcagttctcgttgtcgc

SEQ ID NO: 26 - protein, 159 aa, synthetic construct
MDDAAELRYYEDEDTPGMKTFKGATTGLVAGTLWGAIIATWHDVPRVERSVALPGLIRTLKMMGNHGATFAAIGGVYIGVEQLVQNYRMKRDFVNGAVGGFVAGASILGYKGRSISTAISAGAALAVTSGVIDGLGATTRTDNGKEYYPYTTKKRAAVE